(19) United States
(12) Patent Application Publication
(10) Pub. No.: US 2015/0342483 A1
(43) Pub. Date: Dec. 3, 2015

(54) SYSTEM FOR NON-INVASIVE MEASUREMENT OF INTRACRANIAL PRESSURE AND ASSOCIATED METHODS

(71) Applicant: BIOLOGICAL ILLUMINATION, LLC, Melbourne, FL (US)

(72) Inventors: Fredric S. Maxik, Indialantic, FL (US); David E. Bartine, Cocoa, FL (US); Gregory Flickinger, Indialantic, FL (US); Ran Zhou, Rockledge, FL (US); Matthew Regan, Melbourne, FL (US); Mark Andrew Oostdyk, Cape Canaveral, FL (US)

(73) Assignee: BIOLOGICAL ILLUMINATION, LLC, Melbourne, FL (US)

(21) Appl. No.: 14/728,606

(22) Filed: Jun. 2, 2015

Related U.S. Application Data
(60) Provisional application No. 62/006,455, filed on Jun. 2, 2014.

(51) Int. Cl. A61B 5/03; A61B 5/00; A61B 3/00

(52) U.S. Cl. CPC A61B 5/031; A61B 3/0008; A61B 5/7278; A61B 5/746; A61B 5/6803; A61B 5/742; A61B 5/0082; A61B 5/747; A61B 2562/185

(57) ABSTRACT

An intracranial pressure measuring system comprising a light source, a controller operatively coupled to the light source and a sensor operatively coupled to the controller. The controller is configured to operate the light source to emit light within a wavelength range that can be reflected by ocular vasculature of a person. The sensor is configured to measure a diagnostic reflectance being the intensity of light reflected by the ocular vasculature. The controller is configured to determine if the diagnostic reflectance deviates beyond a threshold and to perform an action responsive to the diagnostic reflectance deviating beyond the threshold.

### RELATED APPLICATIONS

This application is related to and claims priority under 35 U.S.C. §119(e) of U.S. Provisional Patent Application Ser. No. 62/006,455 titled System for Non-Invasive Measurement of Intracranial Pressure filed Jun. 2, 2015, the content of which is incorporated by reference herein in its entirety, except to the extent disclosure therein is inconsistent with disclosure herein.

### FIELD OF THE INVENTION

The present invention relates to systems and methods for non-invasive ocular measurement of intracranial pressure.

### BACKGROUND OF THE INVENTION

Elevated intracranial pressure (ICP) is a leading cause of severe harm to the brain, and is often fatal if very high ICP is not identified and goes untreated for an extended duration. One of the most common causes of increased ICP is head trauma. Head trauma usually occurs in settings in which traditional methods of measuring ICP, such as ultrasound time of flight, Doppler ultrasonography, otoacoustic emission, and optic nerve sheath diameter, are not available. Each of these methods require diagnostic equipment that is either too expensive or too large to be used in setting in which head trauma occurs, such as at sporting events. Accordingly, there is a need in the art for a method capable of measuring ICP in a setting other than those associate with traditional methods, such as in health care facilities, and a device capable of performing such a method.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to an intracranial pressure measuring system comprising a light source, a controller operatively coupled to the light source, and a sensor operatively coupled to the controller. The controller may be configured to operate the light source to emit light within a wavelength range that can be reflected by ocular vasculature of a person. Additionally, the sensor may be configured to measure a diagnostic reflectance being the intensity of light reflected by the ocular vasculature. Furthermore, the controller may be configured to determine if the diagnostic reflectance deviates beyond a threshold. The controller may be configured to perform an action responsive to the diagnostic reflectance deviating beyond the threshold.

In some embodiments, the light source may be configured to emit light having a peak intensity within the range from 620 nm to 750 nm. Additionally, the light source may comprise a light-emitting diode (LED).

Furthermore, the controller may be configured to receive a baseline reflectance from the sensor and determine if the diagnostic reflectance deviates beyond a threshold value from the baseline reflectance. Additionally, the sensor may be configured to provide multiple indications of light reflected from ocular vasculature. The controller may further be configured to determine the baseline reflectance from the multiple indications of light reflected from the ocular vasculature.

In some embodiments, the intracranial pressure measuring system may further comprise a communication device operably coupled to the controller. Furthermore, the action may comprise alerting a medical professional.

In some embodiments, the intracranial pressure measuring system may further comprise a light shield configured to exclude environmental light from the sensor. Additionally, the intracranial pressure measuring system may further comprise an activation device.

In some embodiments, the intracranial pressure measuring system may be incorporated into at least one of glasses, goggles, contact lenses, athletic glasses, athletic goggles, athletic helmets, and hats.

The intracranial pressure measuring system may further comprise a display screen operably coupled to the controller; wherein the action comprises displaying an alert on the display screen.

Additionally, embodiments of the present invention are directed to a method for measuring intracranial pressure using a system comprising a light source, a controller operatively coupled to the light source, and a sensor operatively coupled to the controller. The method may comprise the steps of emitting a diagnostic light, measuring a diagnostic reflectance, determining if the diagnostic reflectance deviates beyond a threshold, and if the diagnostic reflectance deviates beyond the threshold, performing an action. The method may further comprise the steps of emitting a baseline light, measuring a baseline reflectance being the intensity of the baseline light reflected by the ocular vasculature, and determining a baseline reflectance.

In some embodiments, the step of determining if the diagnostic reflectance deviates beyond a threshold may comprise determining if the diagnostic reflectance deviates from the baseline reflectance beyond a threshold. Additionally, the step of emitting a baseline light may comprise emitting multiple baseline lights, the step of measuring a baseline reflectance may comprise measuring the intensity of the multiple baseline lights reflected by the ocular vasculature, and the step of determining a baseline reflectance may comprise analyzing the intensity of the multiple baseline lights reflected by the ocular vasculature.

In some embodiments, the system may further comprise a communication device. The step of performing an action may comprise alerting a medical professional via the communication device.

The method may further comprise the steps of determining if measuring a subsequent diagnostic reflectance is to be taken and upon a determination that a subsequent diagnostic reflectance is to be measured, performing the steps of emitting a subsequent diagnostic light, measuring a subsequent diagnostic reflectance, determining if the subsequent diagnostic reflectance deviates beyond a threshold, and if the subsequent diagnostic reflectance deviates beyond the threshold, performing an action.

### BRIEF DESCRIPTION OF THE DRAWINGS

FIG. 1 is an environmental view of an intracranial pressure measurement system in use according to an embodiment of the invention.

FIG. 2 is a flowchart illustrating a method of measuring intracranial pressure according to an embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention text, as shown and described by the various figures and accompanying text, provides a system for measuring intracranial pressure (ICP) non-invasively by measuring the change in reflection and absorption of light by the ophthalmic artery or any other vasculature as may reflect light emitted onto the eye.

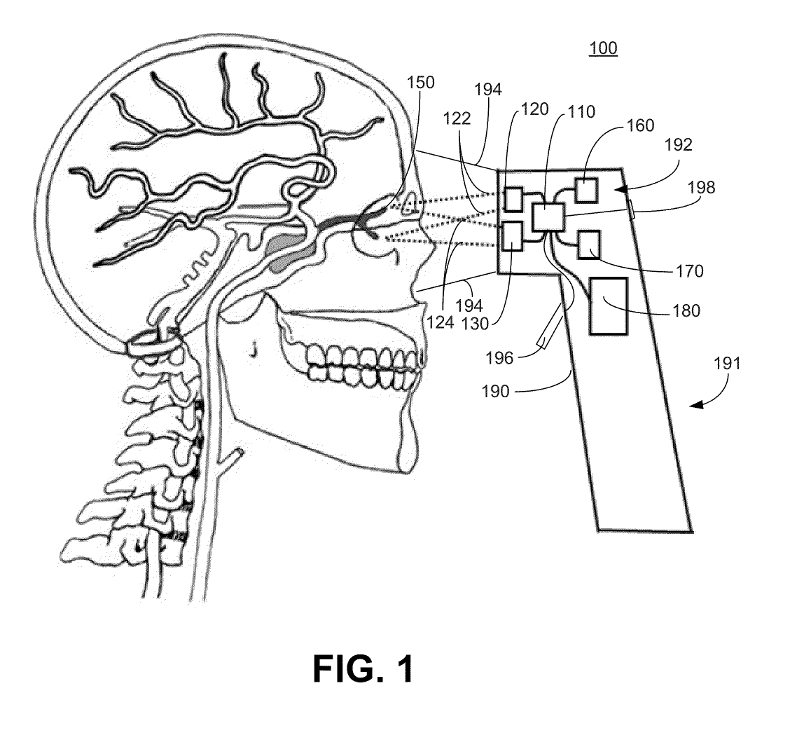

Referring now to FIG. 1, an ICP measuring system 100 according to an embodiment of the invention will be discussed. The ICP measuring system 100 may comprise a controller 110, a light source 120, and a light sensor 130. The controller 110 may be operatively coupled to the light source 120 so as to control the operation of the light source 120. Moreover, the controller 110 may be configured so as to control the operation of the light source 120 to emit light that may enable the measurement of ICP. More specifically, the controller 110 may be configured so as to operate the light source 120 to emit light that may be reflected and/or absorbed by vasculature associated with an eye, such as the ophthalmic artery 150.

The light source 120 may be any lighting device that may emit light within a wavelength range that may be absorbed and/or reflected by the ocular vasculature. More specifically, the light source 120 may be any lighting device that may be operated to emit light within a wavelength range that may be absorbed and/or reflected by at least one of the anatomy of ocular vasculature or a substance, such as hemoglobin, that may pass through the lumen of the ocular vasculature.

In some embodiments, the light source 120 may be configured so as to emit light across the visible spectrum. In some embodiments, the light source 120 may be configured to emit light within a section of the visible spectrum, such as the red spectrum. In such embodiments, the light source 120 may be configured to emit light having a peak intensity within the range from 620 nm to 750 nm. In some embodiments, the light source 120 may comprise one or more light-emitting semiconductors, such as light-emitting diodes (LEDs). Any type of light-emitting device is contemplated and included within the scope of the invention, including incandescents, fluorescents, high-intensity discharge, and the like.

Additionally, the controller 110 may be operatively coupled to the sensor 130. The sensor 130 may be configured to measure the intensity of light incident thereupon. In some embodiments, the sensor 130 may be configured to measure the intensity of light incident thereupon with a specific wavelength range, such as light within a wavelength range from 620 nm to 750 nm or any range therewithin. Additionally, the sensor 130 may be configured to transmit a signal indicating the intensity of light incident thereupon to the controller 110. Each of the light source 120 and the sensor 130 may be configured to emit and measure, respectively, light within a wavelength range that is reflected by the ocular vasculature. More specifically, each of the light source 120 and the sensor 130 may be configured to emit and measure, respectively, light within a wavelength range, the reflectance of which, will vary according to changes in ICP. For example, each of the light source 120 and the sensor 130 may be configured to emit and measure, respectively, light within a wavelength range that may be reflected by hemoglobin in the ocular vasculature, such as the ophthalmic artery 150.

Accordingly, the controller 110 may be configured to operate the light source 120 so as to emit light 122 that may be incident upon the ocular vasculature, such as the ophthalmic artery 150. Reflected light 124 that is incident upon the sensor 130 may have its intensity measured, and the sensor 130 may transmit to the controller 110 an indication of the measured intensity.

In some embodiments, the controller 110 may be configured to determine a baseline ICP. More specifically, the controller 110 may be configured to operate the light source 120 to emit light multiple times, thereby causing the sensor 130 to measure the light reflected from the ocular vasculature multiple times for each instance the light source 120 emits light and transmit an indication of each measurement to the controller 110. The controller 110 may further be configured to analyze the multiple indications transmitted by the sensor 130 so as to determine a baseline intensity of light reflected from the ocular vasculature. Furthermore, the controller 110 may be configured to identify an indication received from the sensor 130 that indicates a significant change in the intensity of light reflected by the ocular vasculature, indicating a significant change in ICP. The controller 110 may further be configured to perform an action responsive to a significant change in the intensity of light reflected by the ocular vasculature. The magnitude of deviation of intensity of light reflected by the ocular vasculature requiring performance of an action may vary between individuals, or there may be a range within which the intensity of reflected light indicates normal ICP, and without which the intensity of reflected light indicates abnormal ICP.

The ICP measuring system 100 may further comprise a memory 160 positioned in communication with the controller 110. The controller 110 may be configured to record the intensity indicated by the sensor 130 to the memory 160 for storage and subsequent retrieval. The memory 160 may be any type of electronic storage device as is known in the art, including, but not limited to, flash memory, or any other type of volatile or non-volatile memory. Additionally, the controller 110 may be configured to utilize the memory 160 to store measurements received by the sensor 130 so as to facilitate the determination by the controller 110 of a baseline intensity. The baseline intensity may further be stored on the memory 160 and accessible by the controller 110 so that the controller 110 may compare an instant measurement from the sensor 130 to determine whether the instant measurement deviates significantly from the baseline intensity.

The ICP measuring system 100 may further comprise a communication device 170. The communication device 170 may be operably coupled to the controller 110. Furthermore, the communication device 170 may be configured to receive a transmission from the controller 110 and may further be configured to transmit the transmission from the controller 110 to a remote computerized device. In some embodiments, the communication device 170 may be configured to transmit the transmission received from the controller 110 across a network. The communication device 170 may be configured to communicate using acoustic communication, visible light communication, IR communication, or radio communication, including protocols such as Wi-Fi, Bluetooth, Zigbee, Rubee, cellular data communication, or any other wireless communication standard as is known in the art. Furthermore, the communication device 170 may be configured to receive transmissions from a remote computerized device and relay the received transmission to the controller 110.

In some embodiments, the ICP measuring system 100 may further comprise a power source 180. The power source 180 may be configured to provide electrical power to the various electrical components of the ICP measuring system 100, including, but not limited to, the controller 110, the light source 120, the sensor 130, the memory 160, and the communication device 170. Furthermore, in some embodiments, the controller 110 may be configured to cooperate with the power source 180 to distribute electrical power to the various electrical components of the ICP measuring system 100.

Furthermore, in the present embodiment, the ICP measuring system 100 may include a housing 190. The housing 190 may be configured to carry the various components of the ICP measuring system 100. Moreover, the housing 190 may be configured to facilitate the operation of the ICP measuring system 100 by an operator. The housing 190 may define an interior 192 within which electrical components of the ICP measuring system 100 may be carried within, so as to protect those components from environmental factors such as precipitation and ambient light. Additionally, the hosing 190 may comprise a lower section 191. The lower section 191 may be configured so as to be graspable by an operator of the ICP measuring system 100 and facilitate the operation thereof.

In some embodiments, the housing 190 may comprise a light shield 194. The light shield 194 may extend from the housing 190 in the direction that the light source 120 emits light. The light shield 194 may be configured so as to generally obscure light from the environment from being incident upon the sensor 130. In some embodiments, the light shield 194 may be configured to interface with the skin of an individual for whom ICP is being measured to further shield the sensor 130 from environmental light.

The housing 190 may further comprise an activation device 196. The activation device 196 may be operably coupled to the controller 110. Furthermore, the activation device 196 may be configured to generate a signal that causes the controller 110 to take a measurement of the light reflected by the ocular vasculature. In the present embodiment, the activation device 196 is a trigger mechanism. Other types of activation devices are contemplated and included within the scope of the invention, including button mechanisms, toggles, touch-sensitive mechanisms, and the like.

The housing 190 may further comprise a display screen 198. The display screen 198 may be operably coupled to the controller 110. Furthermore, the display screen 198 may be configured to present information to an operator responsive to a signal sent from the controller 110. In some embodiments, the controller 110 may be configured to present to the operator whether the intensity of reflected light indicates a normal or abnormal ICP via the display screen 198. In some embodiments, the controller 110 may be configured to present to the operator an ICP as inferred from the intensity of reflected light via the display screen 198. In some embodiments, the controller 110 may be configured to display a warning to the operator if the intensity of reflected light indicates abnormal ICP. The display screen 198 may be any type of display as known in the art, including LCD screens, OLED screens, segmented LCD screens, and the like.

While the present embodiment depicts an ICP measuring system 100 in a handheld embodiment, other implementations of determining ICP by measuring changes in the reflectance of light from ocular vasculature is contemplated. For example, the electrical components, including at least the controller 110, light source 120, and sensor 130 may be incorporated into other structures or articles that may facilitate the positioning of the light source 120 so as to be able to emit light that is incident upon ocular vasculature and the sensor 130 to measure the reflectance of light emitted by the light source 120 thereby. Such other structures or articles include, but are not limited to, glasses, goggles, contact lenses, athletic glasses, athletic goggle, athletic helmets, hats, veils, and the like.

In such embodiments where the ICP measuring system 100 is incorporated into another article such as, for example, a football helmet, those skilled in the art will appreciate that the ICP of the wearer may be continuously monitored. Upon determining that the ICP of the wearer is abnormal, a signal may be sent to a monitoring system (or monitoring station). This may alert a medical professional, for example, that the wearer may be suffering from some side effect of elevated ICP such as, for example, a concussion. Upon receiving such an alert, the medical professional may further examine the wearer to provide appropriate treatment.

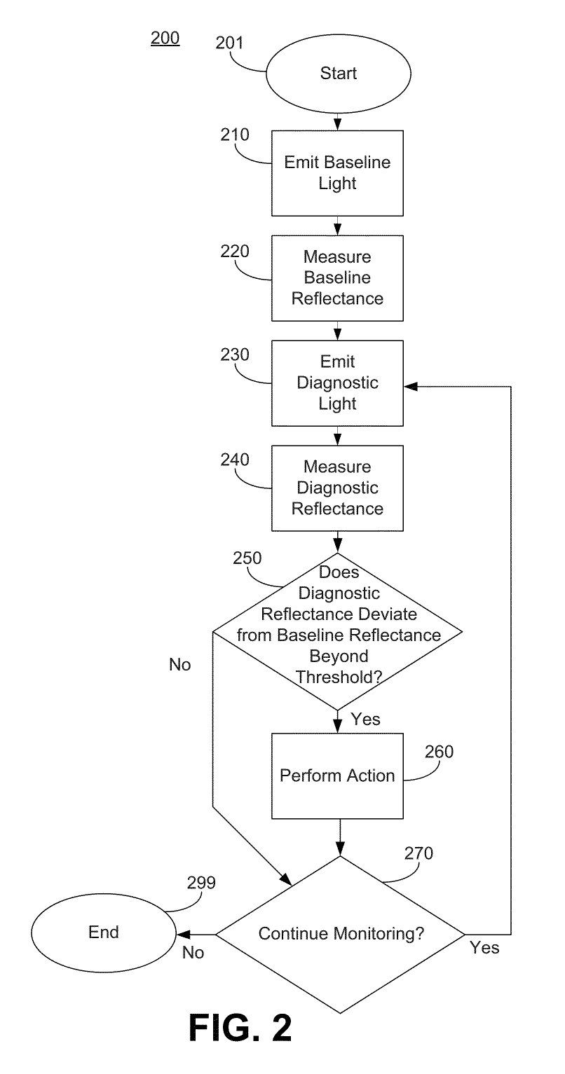

Referring now to FIG. 2, a method 200 according to an embodiment of the invention is presented. Beginning at Block 201, the method 200 continues at Block 210 where a baseline light is emitted so as to be incident upon ocular vasculature. Moreover, the light emitted may be within a wavelength range so as to be reflected by the ocular vasculature. Continuing at Block 220, a baseline reflectance may be measured. The baseline reflectance may indicate an intensity of reflected light indicating a normal ICP.

At Block 230, a diagnostic light may be emitted so as to be incident upon and reflected by ocular vasculature. At Block 240, a diagnostic reflectance may be measured. At Block 250, it may be determined whether the diagnostic reflectance deviates from the baseline reflectance beyond a threshold value. Deviation beyond a threshold value may indicate an abnormal ICP. If it is determined at Block 250 that the diagnostic reflectance does not deviate from the baseline reflectance beyond the threshold value, the method 200 may continue at Block 270 where it is determined whether a subsequent measurement of ICP is to be taken. If a subsequent measurement is not to be taken, the method 200 may end at Block 299. If a subsequent measurement is to be taken, the method 200 may return to Block 230.

If it is determined at Block 250 that the diagnostic reflectance does deviate from the baseline reflectance beyond a threshold value, the method 200 may continue at Block 260 where an action may be taken. The action taken may be any or all of a variety of actions, including, but not limited to, providing at least one of a visual and an auditory alert to an operator of the device performing the measurement, displaying that the diagnostic reflectance is abnormal, displaying an ICP based on the diagnostic reflectance, transmitting a signal indicating at least one of an abnormal reflectance indicating an abnormal ICP and an ICP based on the diagnostic reflectance, and recording at least one of an abnormal reflectance indicating an abnormal ICP and an ICP based on the diagnostic reflectance to a storage device. In some embodiments, where the device performing the measurement of the ICP comprises an imaging device, an image of at least one of an eye or any anatomical component of the eye, such as the vasculature of the eye that may include the ophthalmic artery, with which the diagnostic reflectance is associated may be captured by the imaging device. The captured image may be at least one of stored on a local storage medium and transmitted to a remote computerized device.

The method 200 may continue at Block 270 where it is determined whether a subsequent measurement of ICP is to be taken. If a subsequent measurement is not to be taken, the method 200 may end at Block 299. If a subsequent measurement is to be taken, the method 200 may return to Block 230.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. An intracranial pressure measuring system comprising:
a light source;
a controller operatively coupled to the light source; and
a sensor operatively coupled to the controller;
wherein the controller is configured to operate the light source to emit light within a wavelength range that can be reflected by ocular vasculature of a person;
wherein the sensor is configured to measure a diagnostic reflectance being the intensity of light reflected by the ocular vasculature;
wherein the controller is configured to determine if the diagnostic reflectance deviates beyond a threshold; and
wherein the controller is configured to perform an action responsive to the diagnostic reflectance deviating beyond the threshold.

2. The intracranial pressure measuring system according to claim 1 wherein the light source is configured to emit light having a peak intensity within the range from 620 nm to 750 nm.

3. The intracranial pressure measuring system according to claim 1 wherein the light source comprises a light-emitting diode (LED).

4. The intracranial pressure measuring system according to claim 1 wherein the controller is configured to:
receive a baseline reflectance from the sensor; and
determine if the diagnostic reflectance deviates beyond a threshold value from the baseline reflectance.

5. The intracranial pressure measuring system according to claim 4 wherein the sensor is configured to provide multiple indications of light reflected from ocular vasculature; and wherein the controller is configured to determine the baseline reflectance from the multiple indications of light reflected from the ocular vasculature.

6. The intracranial pressure measuring system according to claim 1 further comprising a communication device operably coupled to the controller.

7. The intracranial pressure measuring system according to claim 6 wherein the action comprises alerting a medical professional.

8. The intracranial pressure measuring system according to claim 1 further comprising a light shield configured to exclude environmental light from the sensor.

9. The intracranial pressure measuring system according to claim 1 further comprising an activation device

10. The intracranial pressure measuring system according to claim 1 wherein the intracranial pressure measuring system is incorporated into at least one of glasses, goggles, contact lenses, athletic glasses, athletic goggles, athletic helmets, and hats.

11. The intracranial pressure measuring system according to claim 1 further comprising a display screen operably coupled to the controller; wherein the action comprises displaying an alert on the display screen.

12. A method for measuring intracranial pressure using a system comprising a light source, a controller operatively coupled to the light source, and a sensor operatively coupled to the controller, the method comprising the steps of:
emitting a diagnostic light;
measuring a diagnostic reflectance;
determining if the diagnostic reflectance deviates beyond a threshold; and
if the diagnostic reflectance deviates beyond the threshold, performing an action.

13. The method according to claim 12 further comprising the steps of:
emitting a baseline light;
measuring a baseline reflectance being the intensity of the baseline light reflected by the ocular vasculature; and
determining a baseline reflectance.

14. The method according to claim 13 wherein the step of determining if the diagnostic reflectance deviates beyond a threshold comprises determining if the diagnostic reflectance deviates from the baseline reflectance beyond a threshold.

15. The method according to claim 13 wherein:
the step of emitting a baseline light comprises emitting multiple baseline lights;
the step of measuring a baseline reflectance comprises measuring the intensity of the multiple baseline lights reflected by the ocular vasculature; and
the step of determining a baseline reflectance comprises analyzing the intensity of the multiple baseline lights reflected by the ocular vasculature.

16. The method according to claim 12 wherein the system further comprises a communication device; and wherein the step of performing an action comprises alerting a medical professional via the communication device.

17. The method according to claim 12 further comprising the steps of:
determining if measuring a subsequent diagnostic reflectance is to be taken; and
upon a determination that a subsequent diagnostic reflectance is to be measured, performing the steps of:
emitting a subsequent diagnostic light,
measuring a subsequent diagnostic reflectance,
determining if the subsequent diagnostic reflectance deviates beyond a threshold, and
if the subsequent diagnostic reflectance deviates beyond the threshold, performing an action.

18. A method for measuring intracranial pressure using a system comprising a light source, a controller operatively coupled to the light source, and a sensor operatively coupled to the controller, the method comprising the steps of:
emitting multiple baseline lights;
measuring the intensity of the multiple baseline lights reflected by the ocular vasculature;
analyzing the intensity of the multiple baseline lights reflected by the ocular vasculature to determine a baseline reflectance;
emitting a diagnostic light;
measuring a diagnostic reflectance;
determining if the diagnostic reflectance deviates from the baseline reflectance beyond a threshold; and
if the diagnostic reflectance deviates from the baseline reflectance beyond the threshold, performing an action.

19. The method according to claim 18 wherein the system further comprises a communication device; and wherein the step of performing an action comprises alerting a medical professional via the communication device.

20. The method according to claim 18 further comprising the steps of:
determining if measuring a subsequent diagnostic reflectance is to be taken; and
upon a determination that a subsequent diagnostic reflectance is to be measured, performing the steps of:
emitting a subsequent diagnostic light,
measuring a subsequent diagnostic reflectance,
determining if the subsequent diagnostic reflectance deviates beyond a threshold, and
if the subsequent diagnostic reflectance deviates beyond the threshold, performing an action.
